(19) United States
(12) Patent Application Publication
(10) Pub. No.: US 2008/0161675 A1
(43) Pub. Date: Jul. 3, 2008

(54) Ultra-Short Mri Body Coil

(75) Inventors: Paul R. Harvey, Best (NL); Eerke Holle, Riethoven (NL); Zhiyong Zhai, Cleveland, OH (US)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/817,968

(22) PCT Filed: Mar. 3, 2006
(86) PCT No.: PCT/IB06/50679

§ 371 Date: Sep. 7, 2007

Related U.S. Application Data
(60) Provisional application No. 60/660,188, filed on Mar. 10, 2005.

(51) Int. Cl. A61B 5/055

(52) U.S. Cl. 600/410

(57) ABSTRACT

A magnetic resonance imaging system (10) utilizes an ultra-short RF body coil (36). The ultra-short body coil (36) is shorter than the mechanical equivalent birdcage coil by at least a factor of two. Such coil provides equivalent (Bₜ) magnetic field-uniformity, while conforming to SAR limitations.

The following relates to the magnetic resonance arts. It finds particular application in magnetic resonance imaging coils and scanners, and will be described with particular reference thereto. More generally, it finds application in magnetic resonance systems for imaging, spectroscopy, and so forth.

In MRI, RF coils are used to generate B₁ magnetic fields within the imaging subject for exciting the nuclear spins and detecting signals from the nuclear spins. High frequency body coils (128 MHz) which operate at 3.0 T and above, are required to operate efficiently homogenously, and to meet the Specific Absorption Rate (SAR) regulations. The SAR regulations represent the RF dosimetry quantification of the magnitude and distribution of absorbed electromagnetic energy within biological subjects that are exposed to the RF fields.

Current approach for the high frequency body coil is to build a shielded birdcage coil. The birdcage coil has multiple conductor rungs which are arranged around the examination region extending parallel to the main field direction. The parallel conductor rungs are connected to each other via an end cap or ring at one end of the coil and a circular loop conductor at the other end. Typically, the whole body birdcage coil is 40 cm-60 cm in length for a 40 cm field of view. Current flows back and forth through the rungs, the end cap, and the loop. Birdcage coils exhibit a substantially uniform magnetic field distribution in the interior at frequencies at or under 128 MHz, which correspond to proton imaging in a main B₀ magnetic field of 3 T.

However, for super high field applications (B₀>3 T), the application of the birdcage coils is limited with respect to radiation losses due to propagation effects inside the bore of the MR system and strong loading effects of the tissue. Typically, the losses become unacceptable when half the wavelength at resonance is less than the bore diameter. The problem of radiation losses can be overcome by reducing the diameter of the RF bore or shortening the length of the birdcage coil. However, reducing the coil length reduces the coil efficiency and homogeneity over the desired filed of view. End-ring components generate a B₁ field component which coupled to the main B₀ magnetic field. Reducing the diameter of the bore increases the cut off frequency, but the strong coupling to the tissue due to RF eddy currents (of) is still a fundamental problem. The induced impedance in the conductors caused by the asymmetric subject loading can generate strong B₁ inhomogeneity.

To solve the efficiency, homogeneity and frequency problems associated with the end-ring-dependent birdcage coils, the TEM coils can be used as body coils. The TEM coil typically includes parallel resonators, which are arranged around the examination region. The TEM coil is typically open on both ends, lacking both the end cap and the circular loop conductor. TEM coils provide improved radio frequency performance compared with the birdcage coils for higher frequencies corresponding to B₀>3 T. The TEM coil of a given length can be built to the large diameters, without significantly changing the frequency of the coil.

However, currently used TEM coils at 3.0 T and higher do not meet the SAR requirements.

The following contemplates improved apparatuses and methods that overcome the aforementioned limitations and others.

According to one aspect, a magnetic resonance imaging system is disclosed. A cylindrical magnet generates a substantially uniform main magnetic field through an examination region. A cylindrical ultra-short radio frequency body coil is disposed coaxially with the magnet to generate radio frequency excitation pulses in the examination region, which ultra-short body coil conforms to Specific Absorption Rate (SAR) limitations.

According to another aspect, a method of magnetic resonance imaging is disclosed. A substantially uniform main magnetic field through an examination region is generated with a magnet. Radio frequency excitation pulses in the examination region are generated with an ultra-short radio frequency body coil which conforms to Specific Absorption Rate (SAR) limitations.

One advantage resides in reducing the SAR.

Another advantage resides in providing a system with more openness.

Numerous additional advantages and benefits will become apparent to those of ordinary skill in the art upon reading the following detailed description.

The invention may take form in various components and arrangements of components, and in various process operations and arrangements of process operations. The drawings are only for the purpose of illustrating preferred embodiments and are not to be construed as limiting the invention.

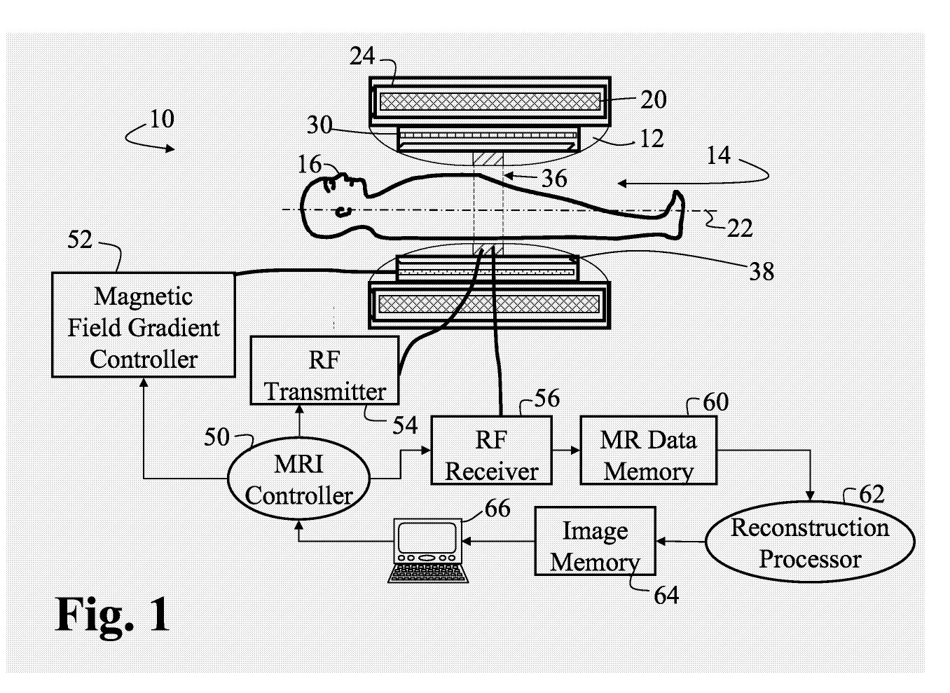

FIG. 1 diagrammatically shows a magnetic resonance imaging system employing a radio frequency coil;

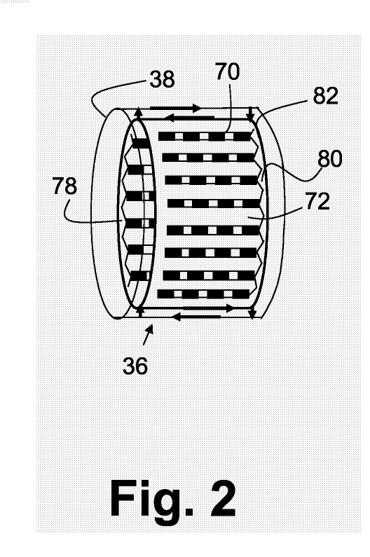

FIG. 2 shows a perspective view of the radio frequency coil of FIG. 1;

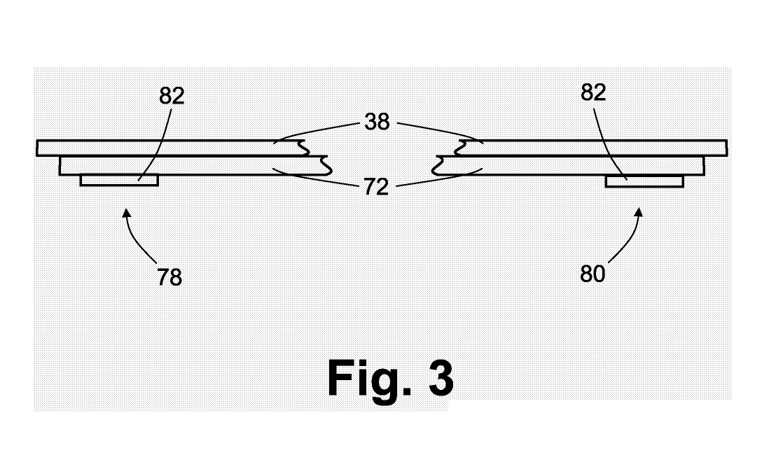

FIG. 3 shows a cross-section of the strip-type end-rings and adjacent portions of the generally cylindrical substrate;

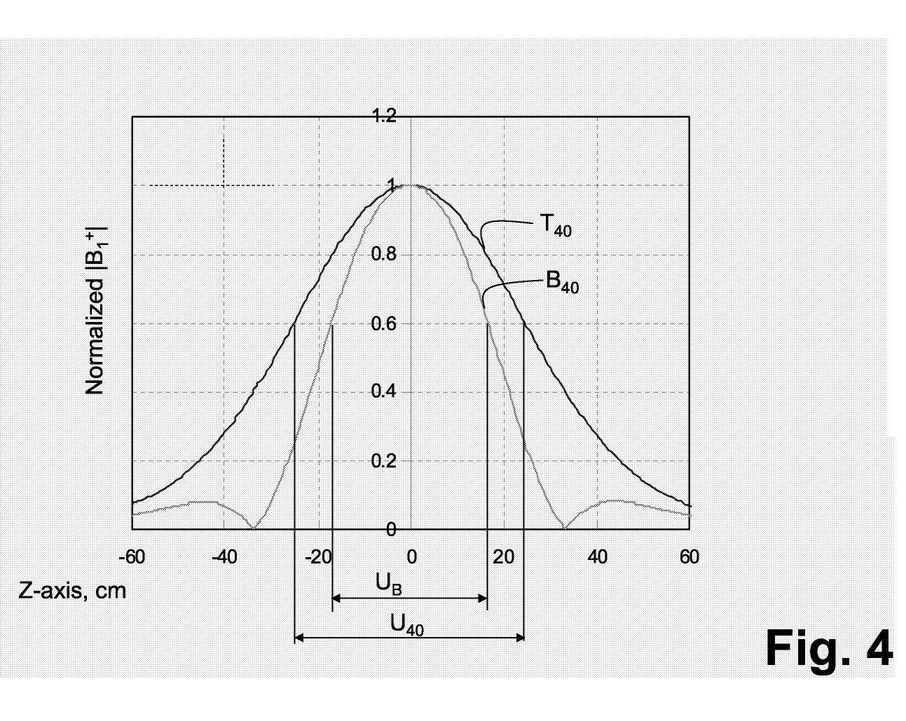

FIG. 4 shows graphs of a normalized B₁ magnetic field versus z-axis for a TEM coil and a birdcage coil;

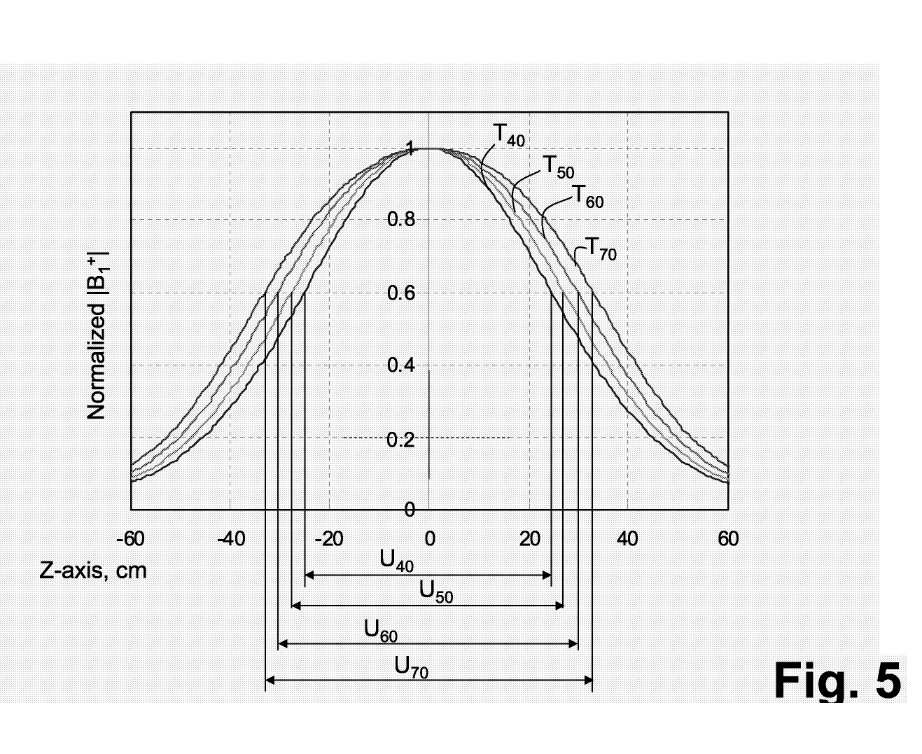

FIG. 5 shows graphs of a normalized B₁ magnetic field versus z-axis for a TEM coil of different lengths;

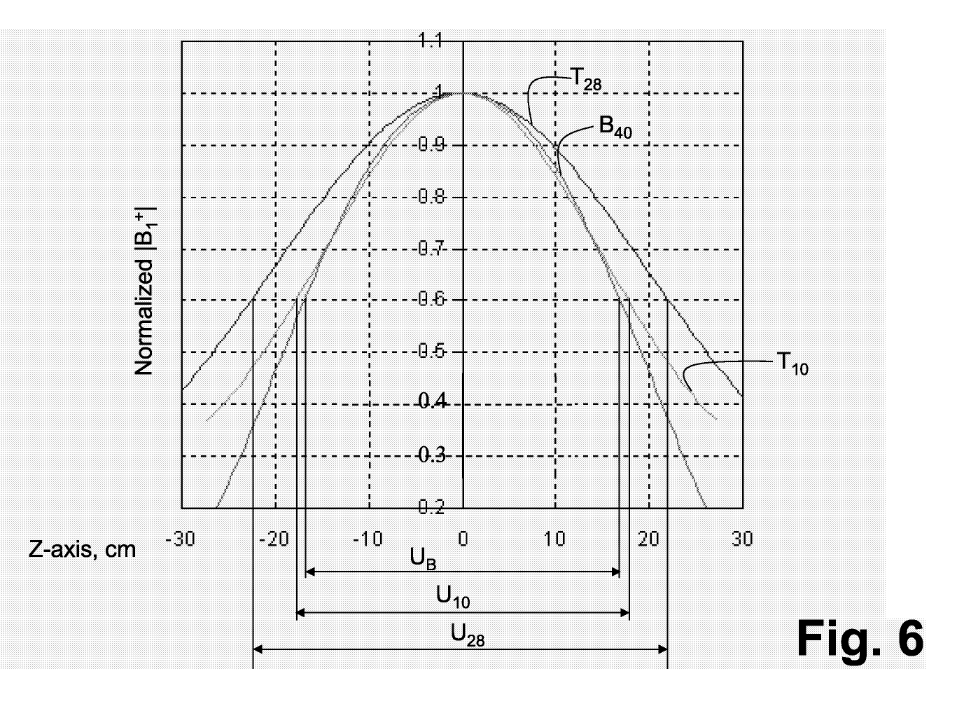

FIG. 6 shows a graph of a normalized B₁ magnetic field versus z-axis for a TEM coil with 28 cm and 10 cm lengths and for a birdcage coil with a 40 cm length;

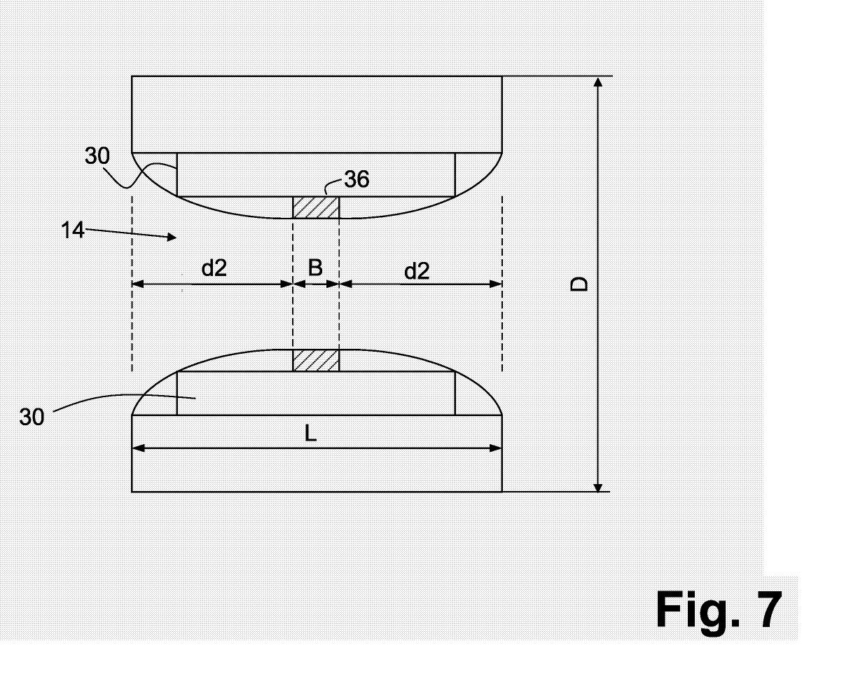

FIG. 7 shows a bore of an MRI scanner of FIG. 1, which employs an ultra-short RF coil; and

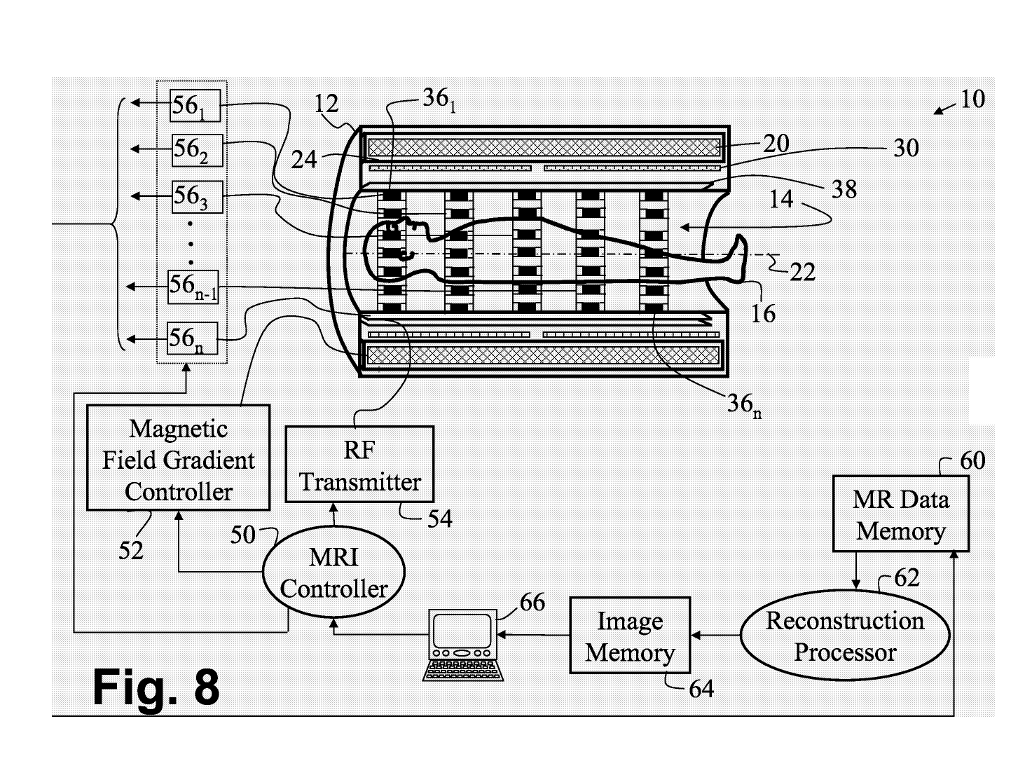

FIG. 8 diagrammatically shows a magnetic resonance imaging system employing more than one radio frequency coils.

With reference to FIG. 1, a magnetic resonance imaging scanner 10 includes a housing 12 defining an examination region 14 in which is disposed a patient or other imaging subject 16. A main magnet 20 disposed in the housing 12 generates a main magnetic field in the examination region 14. Typically, the main magnet 20 is a superconducting magnet surrounded by cryoshrouding 24; however, a resistive main magnet can also be used. Magnetic field gradient coils 30 are arranged in or on the housing 12 to superimpose selected magnetic field gradients on the main magnetic field within the examination region 14. A whole-body radio frequency coil 36, such as an ultra-short RF body coil with a surrounding shield 38, is disposed about the examination region 16. Preferably, the coil 36 is a transmission line ring TEM coil as described in detail below. Of course, it is also contemplated that the coil 36 may be a TEM coil, a hybrid TEM coil, or the like. The coil 36 is preferably circularly cylindrical, but, of course, might have other geometries, such as an elliptic cross-section, semi-circular cross-section, semi-elliptical cross-section, and the like.

With continuing reference to FIG. 1, a magnetic resonance imaging controller 50 operates magnetic field gradient controllers 52 coupled to the gradient coils 30 to superimpose selected magnetic field gradients on the main magnetic field in the examination region 14, and also operates radio frequency transmitters 54 coupled to the radio frequency coil 36 to inject selected radio frequency excitation pulses at about the magnetic resonance frequency into the examination region 14. Preferably, each rung of the coil is independently driven. The radio frequency excitation pulses excite magnetic resonance signals in the imaging subject 16 that are spatially encoded by the selected magnetic field gradients. Still further, the imaging controller 50 operates radio frequency receivers 56 also connected with the radio frequency coil 36 to demodulate the generated and spatially encoded magnetic resonance signals. Preferably, each rung is connected to a different receive channel. The received spatially encoded magnetic resonance data is stored in a magnetic resonance data memory 60.

A reconstruction processor 62 reconstructs the stored magnetic resonance data into a reconstructed image of the imaging subject 16 or a selected portion thereof lying within the examination region 14. The reconstruction processor 62 employs a Fourier transform reconstruction technique or other suitable reconstruction technique that comports with the spatial encoding used in the data acquisition. The reconstructed image is stored in an images memory 64, and can be displayed on a user interface 66, transmitted over a local area network or the Internet, printed by a printer, or otherwise utilized. In the illustrated embodiment, the user interface 66 also enables a radiologist or other user to interface with the imaging controller 50 to select, modify, or execute imaging sequences. In other embodiments, separate user interfaces are provided for operating the scanner 10 and for displaying or otherwise manipulating the reconstructed images.

The described magnetic resonance imaging system is an illustrative example. In general, substantially any magnetic resonance imaging scanner can incorporate the disclosed radio frequency coils. For example, the scanner can be an open magnet scanner, a vertical bore scanner, a low-field scanner, a high-field scanner, or so forth. In the embodiment of FIG. 1, the radio frequency coil 36 is used for both transmit and receive phases of the magnetic resonance sequence; however, in other embodiments separate transmit and receive coils may be provided, one or both of which may incorporate one or more of the radio frequency coil designs and design approaches disclosed herein. Where more than one body coil is incorporated, the body coils 36 are preferably distributed evenly in the examination region 14. The RF body coil(s) 36 are driven using a quadrature excitation. Alternatively, the RF body coil(s) 36 are driven using four port excitation.

With reference to FIG. 2, the example illustrated radio frequency body coil is a transmission line ring TEM coil 36 (not to scale) which includes a plurality of rungs 70. The rungs 70 are arranged in parallel to one another to surround the examination region 14. In the illustrated coil 36, the rungs 70 include printed circuit segments disposed on an electrically non-conducting generally cylindrical substrate 72, with the printed circuit segments of the rungs 70 connected by lumped capacitive elements (not shown). However, in other embodiments the rungs may be continuous printed circuit segments, continuous free-standing conductors, free-standing conductor segments connected by lumped capacitive elements or conductive traces, transmission lines including overlapping printed circuitry disposed on both the inside and the outside of the generally cylindrical substrate 72, or other types of conductor arrangements. The segments are each capacitively coupled to the RF shield 38.

Two generally annular end-rings 78, 80 are disposed generally transverse to the parallel rungs 70. The end-rings 78, 80 are connected to the rungs 70. A length of the end-ring between two neighboring rungs 70 is selected to provide a selected transmission delay. In general, the selected length is greater than a circumferential arc length between the neighboring rungs 70.

With continuing reference to FIG. 2 and further reference to FIG. 3, the end-rings 78, 80 and rungs 70 include a conductor layer 82 disposed on the inside of the generally cylindrical substrate 72. The conductor layer 82 defines a closed-loop transmission line.

The layout of the conductor layer 82 of the end-rings 78, 80 can have various shapes that satisfy the desired transmission line characteristics such as characteristic impedance, transmission delay, current distribution, and power dissipation. The end-rings 78, 80 can employ certain time shapes with directional components transverse to the annular parameter of the generally annular end-rings to provide a designed extended length between neighboring rungs. The designed extended lengths enable tailoring of the transmission delay and other transmission line characteristics and enable tailoring of the coupling of the end-rings with the rungs through tailoring of transmission line parameters such as a transmission delay and characteristic impedance. This approach eliminates the need for capacitive elements in the end-rings and eliminates the need for capacitive coupling (when compared to a low pass or low pass-like bent pass birdcage configuration) between the end-rings and the rungs.

In the illustrated coil 36, the printed circuitry defining the end-rings 78, 80 and the rungs 70 are directly coupled. In other embodiments, the coupling at the magnetic resonance frequency can be achieved via lumped capacitive elements or via capacitive gaps between the end-rings 78, 80 and the ends of the rungs 70. Kirchoff's law should be satisfied at the intersection of the rings and rungs.

The radio frequency shield 38 is generally cylindrical in shape and is arranged concentrically outside of the arrangement of rungs 70 and outside of the generally cylindrical substrate 72 to define the ground plane of end-ring transmission lines. The generally annular end-rings 78, 80 are arranged coaxially with the generally cylindrical radio frequency shield 38. In one embodiment, the radio frequency shield 38 is spaced apart from the radio frequency coil 36 by electrically non-conductive spacer element (not shown).

With continuing reference to FIG. 1, the RF body coil 36 is significantly shorter than the mechanically equivalent birdcage. More specifically, for equivalent of the B₁ magnetic field uniformity the RF coil 36 is shorter by at least a factor of two compared to the conventional birdcage coil with the same B₁ magnetic field uniformity.

With reference to FIG. 4, graphs T₄₀, B₄₀ of a normalized |B₁⁺|-field versus z-axis in central coronal plane for respective 40 cm long TEM quadrature body coil (QBC) and 40 cm long birdcage QBC is shown. As seen in FIG. 4 and Table 1 below, an RF uniformity for the 40 cm long TEM coil is significantly better than an RF uniformity of the 40 cm long birdcage coil. For instance, the 60% uniformity extends for approximately U₄₀=50 cm in the Z-direction for the TEM coil, while the 60% uniformity extends only for approximately U_{B}=30 cm for the birdcage coil.

TABLE 1

| | | | | Max. Local | |
|---|---|---|---|---|---|
| | Whole | Partial- | | SAR per 10 g | Max. Local |
| | body | body | Head | tissue in | SAR per 10 g |
| | SAR | SAR | SAR | Extremities | tissue in |
| QBC | (W/kg) | (W/kg) | (W/kg) | (W/kg) | Trunk (W/kg) |
| TEM | 1.7 | 2.6 | 0.5 | 13.4 | 13.1 |
| Birdcage | 0.8 | 1.6 | 0.02 | 13.4 | 10 |
| Difference | +53% | +38% | — | 0% | +24% |
| (%) | | | | | |

However, as seen in Table 1, the SAR measurements for the TEM coil are higher in all aspects compared to the SAR measurements of the birdcage of the same length, e.g. 40 cm length coils in this example. For the SAR purposes it becomes disadvantageous to have a more uniform body coil extending over a large region. However, as discussed below, the TEM body coil can be designed of the length which is significantly less than the length of the B₁ field equivalent birdcage coil to conform to the SAR regulations.

With reference to FIG. 5, graphs T₄₀, T₅₀, T₆₀, T₇₀ of a normalized |B₁⁺|-field versus Z-axis in the central transverse plane for TEM QBC are shown. The graphs T₄₀, T₅₀, T₆₀, T₇₀ correspond to the TEM coils with respective coil lengths of 40 cm, 50 cm, 60 cm and 70 cm.

With continuing reference to FIG. 5, for a 40 cm long TEM coil (Graph T₄₀), the region U₄₀ of 60% uniformity extends for approximately 50 cm in the Z-direction. For a 60 cm long TEM coil (Graph T₅₀), the equivalent region U₅₀ extends for approximately 60 cm in the Z-direction. As seen in FIG. 4, the standard 40 cm long birdcage coil has a 60% uniformity region B₄₀ which extends approximately 30 cm in the Z-direction. For the equivalent B₁ magnetic field uniformity, the TEM coil can be shorter, by at least a factor of two, compared to a conventional birdcage coil of 40 cm which has the same B₁ magnetic field uniformity.

Of course, it is also contemplated that the TEM body coil can be of another shorter length as compared to another mechanically equivalent birdcage coil as long as the body coil has an equivalent B₁ magnetic field uniformity and conforms to the SAR limitations. For example, the body coil can be from about 30 cm to about 50 cm long.

With reference to FIG. 6, graphs T₂₈, T₁₄ of a normalized |B₁⁺|-field versus z-axis in the central transverse plane for TEM QBC with respective coil lengths of 28 cm and 10 cm are compared to the graph B₄₀ of the normalized |B₁⁺|-field for the birdcage design of the length equal to 40 cm. As seen in FIG. 6, the TEM body coil of the length equal to 10 cm realizes a B₁ magnetic field uniformity approximately equivalent to the birdcage coil of the length equal to 40 cm.

Table 2 compares SAR measurements for the 10 cm TEM coil and 40 cm birdcage coil. The comparison shows that the 10 cm TEM coil yields approximately the RF uniformity and SAR of the 40 cm birdcage coil. E.g., when the B₁ magnetic field uniformity of the TEM coil is equivalent to that of the birdcage coil, the SAR performance is also very similar.

TABLE 2

| | whole body | | |
|---|---|---|---|
| | SAR | local SAR in | |
| | 100% duty | extremities | local SAR in trunk |
| Coil Type | cycle | 100% duty cycle | 100% duty cycle |
| 10 cm TEM QBC | 86 W/kg | 882 W/kg | 595 W/kg |
| 40 cm Birdcage | 50 W/kg | 834 W/kg | 621 W/kg |

Since local SAR is primarily the limiting factor at higher frequencies of approximately 128 MHz, the fact that the whole-body SAR is slightly higher for the 10 cm TEM than for the birdcage, is not a significant limitation. The TEM coil of the length equal to or less than 20 cm can replace the birdcage coil of the length equal to 40 cm by both conforming to the B₁ magnetic field uniformity requirements and the SAR limitations.

With reference again to FIG. 7, the magnet of the total length L equal to 1.6 m and diameter D equal to 1.9 m, is shown. In this example, the ultra-short RF body coil 36, has a length B equal to 20 cm which occupies only 12.5% of the length of the bore. As shown in FIG. 6, a 20 cm TEM RF coil has a 60% or better uniformity over about 40 cm. This leaves open space of twice distance d2, which is equal to 2×70 cm. Such short RF coil enables greater flaring of the patient bore, which yields a more open look to the system making it more patient friendly, yet provides a bigger field of view. Preferably, the ratio of the ultra-short body coil length B to the main magnet length L is less than 0.16. The 20 cm body coil exhibits approximately the same SAR performance as the 40 cm long birdcage coil. A bigger field of view with higher SAR can be provided with the body coil of a larger length. Lower SAR with a shorter field of view can be provided by a shorter coil.

In one embodiment, the 24 cm body coil is split into two 12 cm coils in the Z-direction to increase flexibility.

With reference to FIG. 8, the magnetic resonance scanner 10 includes two or more ultra-short body coils 36₁, . . . , 36ₙ. The body coils 36₁, . . . , 36ₙ are arranged coaxially with the main magnet 20 and, preferably, distributed evenly in the examination region 14. Magnetic resonance signals are induced in selected ultra-short body coils 36₁, . . . , 36ₙ in the examination region 14. In one embodiment, each of ultra-short body coils 36₁, . . . , 36ₙ is connected with the individual RF receiver 56₁, . . . , 56ₙ. In another embodiment, each ultra-short body coils 36₁, . . . , 36ₙ is connected with the individual transmitter (not shown).

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. A magnetic resonance imaging system comprising:
a cylindrical magnet for generating a substantially uniform main magnetic field through an examination region; and
a cylindrical ultra-short radio-frequency body coil, which is disposed coaxially with the magnet for generating radio frequency excitation pulses in the examination region, which ultra-short body coil conforms to Specific Absorption Rate (SAR) limitations.

2. The system as set forth in claim 1, wherein a ratio of an ultra-short body coil length to a magnet length is equal to or less than 0.16.

3. The system as set forth in claim 1, wherein the body coil has a length to diameter ratio less than 1:2.

4. The system as set forth in claim 1, wherein the body coil has a length to diameter ratio less than 2:5.

5. The system as set forth in claim 1, wherein the ultra-short body coil is at least one of a transverse electromagnetic (TEM) coil and a hybrid including a TEM coil.

6. The system as set forth in claim 1, wherein the ultra-short body coil includes a plurality of resonators disposed circumferentially around the examination region.

7. The system as set forth in claim 6, wherein the resonators are independently driven in a transmit mode.

8. The system as set forth in claim 6, the resonators are each connected with a different receiver channel in a receive mode.

9. The system as set forth in claim 6, wherein the resonators are driven using one of four port excitation and quadrature excitation.

10. The system as set forth in claim 1, wherein the ultra-short body coil includes:
an arrangement of substantially parallel rungs, each rung extending in a direction parallel to a longitudinal direction of the magnet; and
one or more generally annular strip-type end-rings disposed generally transverse to the parallel rungs and connected with the rungs, each generally annular strip-type end-ring being disposed about a cylindrical dielectric layer; and
a radio frequency shield substantially surrounding the arrangement of substantially parallel rungs, the end-rings being coupled with the radio frequency shield.

11. The system as set forth in claim 10, wherein the rungs and end-rings are disposed on an inner perimeter of the cylindrical dielectric layer and the radio frequency shield is disposed on an outer surface of the cylindrical dielectric layer.

12. The system as set forth in claim 10, wherein each rung is an independently tuned resonator.

13. The system as set forth in claim 12, wherein each rung of the ultra-short body coil is driven independently via a channel of a transmitting system to selectively inject RF excitation pulses into the examination region.

14. The system as set forth in claim 12, wherein each rung of the ultra-short body coil is an independent receiving element which is connected to a channel of a receiver to demodulate received MR signals.

15. The system as set forth in claim 1, further including:
two or more ultra-short body coils arranged coaxially and distributed along the examination region.

16. The system as set forth in claim 15, wherein each ultra-short body coil is an independent coil, each coil being connected to an individual RF transmitter, which selectively injects RF excitation pulses into the examination region, and to an individual RE receiver which demodulates and converts MR signals.

17. A method of magnetic resonance imaging comprising:
generating a substantially uniform main magnetic field through an examination region with a magnet; and
generating radio frequency excitation pulses in the examination region with an ultra-short radio frequency body coil which conforms to Specific Absorption Rate (SAR) limitations.

18. The method as set forth in claim 17, wherein a ratio of an ultra-short body coil length to a magnet length is equal to or less than 0.16.

19. The method as set forth in claim 17, further including:
distributing two or more ultra-short body coils along the examination region; and
using the ultra-short body coils simultaneously.

20. A magnetic resonance scanner to perform the method of claim 17.

21. An ultra-short radio frequency body coil, which produces a uniform magnetic field at least at 3.0 T while conforming to Specific Absorption Rate regulations, the coil including:
an arrangement of substantially parallel rungs each functioning as a resonator, the rungs being disposed in parallel around a cylinder, which cylinder has a diameter to length ratio of 2:1 or less;
one or more generally annular strip-type end-rings disposed generally transverse to the parallel rungs and connected with the rungs; and
a radio frequency shield substantially surrounding the arrangement of substantially parallel rungs.

22. A magnetic resonance scanner for use with the coil of claim 21.
